(19) United States
(12) Patent Application Publication
(10) Pub. No.: US 2018/0104435 A1
(43) Pub. Date: Apr. 19, 2018

(54) OXYGEN SUPPLY DEVICE, OXYGEN SUPPLY SYSTEM AND METHOD OF OXYGEN SUPPLY

(71) Applicant: LINDE AKTIENGESELLSCHAFT, München (DE)

(72) Inventors: Sabine Häussermann, Neuried (DE); Peter Forsberg, Ottobrunn (DE)

(73) Assignee: Linde AG, München (DE)

(21) Appl. No.: 15/562,604

(22) PCT Filed: Apr. 1, 2016
(86) PCT No.: PCT/EP2016/057269

§ 371 Date: Sep. 28, 2017

(30) Foreign Application Priority Data
Apr. 2, 2015 (EP) 15162501.9

(51) Int. Cl. A61M 16/10; A61M 16/00; A61M 16/06

(52) U.S. Cl. CPC A61M 16/1005; A61M 16/022; A61M 16/0672; A61M 2202/0208; A61M 2205/8256; A61M 2205/3303; A61M 2205/3569; A61M 2205/825; A61M 2205/3334

(57) ABSTRACT

A clinical oxygen supply device (100) adapted to supply supplemental oxygen to a patient, comprising an oxygen inlet section (110) with a first coupling unit (111) adapted to be releasably coupled to a standardized oxygen wall outlet (1), an oxygen outlet section (120) with a second coupling unit (121) adapted to be releasably coupled to an oxygen dispensing device (2) attached to a patient (3), and an oxygen flow control section (130) with a regulation unit (132) adapted to regulate an oxygen flow between the oxygen inlet section (110) and the oxygen outlet section (120) on the basis of a control signal is provided. Display means adapted to indicate at least one operating parameter of the clinical oxygen supply device, an energy conversion section (140) adapted to generate electrical energy from pressure energy of oxygen supplied to the oxygen inlet section (110), and an energy supply section (150) adapted to provide the electrical energy generated by the energy conversion section (140) to the oxygen flow control section (130) and the display means are provided. The clinical oxygen supply device (100) is configured as a battery-less device. A clinical oxygen supply system (1) and a method of clinical oxygen supply are also part of the present invention.

The invention relates to a clinical oxygen supply device, to a clinical oxygen supply system, and to a method of clinical oxygen supply according to the precharacterising clauses of the independent claims.

### PRIOR ART

Oxygen supply arrangements for clinical purposes may comprise an oxygen wall outlet to which a mechanical oxygen flow setting device is coupled. The mechanical oxygen flow setting device is classically equipped with and based on a so called rotameter, i.e. a device measuring the flow rate of oxygen gas in a closed tube as e.g. originally described in DE 215 225 A, or with another mechanical flow rate indicator.

While, for extensive surgical treatment, a patient in need of oxygen supply is typically intubated and exclusively supplied with oxygen by means of a sophisticated respiratory unit, also termed “ventilator”, this is typically not the case in emergency rooms, during recovery of acute respiratory patients and during some forms of critical care treatment where patients are not completely ventilated but still need supplemental oxygen. In the latter cases, patients are typically supplied with the supplemental oxygen directly from the wall outlet under control of the mechanical oxygen flow setting device described above. If at all, only comparatively simple respiratory units are used in such cases, e.g. devices for providing so called bilevel positive airway pressure (BiPAP) or continuous positive airway pressure (CPAP). However, under supplemental oxygen supply without sophisticated ventilators also (smaller) surgical interventions may be performed, especially under local anaesthetics.

If supplemental oxygen is supplied to a patient, the oxygen saturation of the blood of the patient can be measured. Especially in cases of supplemental oxygen supply over longer periods of time, oxygen is conventionally measured intermittently. Oxygen measurement is typically performed non-invasively via a so called oximeter. In oximeters, typically a sensor is placed on a thin and therefore optically translucent part of the patient's body, usually a fingertip or an earlobe, and light of two wavelengths is passed through this body part to a photodetector. The absorbance of each of the two wavelengths is measured, allowing a determination of the absorbance of the pulsing arterial blood alone without interference of peripheral blood. By measuring the oxygen saturation of the blood of the patient, oxygen dosage can be tailored to specific needs of sensitive patient groups, as explained below. Doing so, over- and underdosing of oxygen can be reliably avoided. However, for patient groups less sensitive to excessive oxygen, it may also be sufficient to only provide a fixed oxygen flow without feedback from a manual or automatic measurement of the oxygen saturation in the patient. The present application relates to both scenarios.

On the basis of an indication of an oxygen saturation sensing device, if a measurement is performed, a nurse or other personnel responsible for the oxygen supply, in the classical arrangements mentioned, manually regulates the oxygen flow to the patient by turning a valve of the mechanical oxygen flow setting device.

From U.S. Pat. No. 6,142,149 A, a mobile system providing an automatically regulated oxygen supply to a patient based on the oxygen saturation in the blood of the patient is known, comprising an automatically operable oxygen flow setting device carried by the patient. U.S. Pat. No. 5,365,922 A, U.S. Pat. No. 6,470,885 A, US 2013/0125891 A1 and U.S. Pat. No. 6,532,958 A also disclose automatically operable oxygen flow setting devices. While, thus, also automatically operable oxygen flow setting devices are known from the prior art, they have never found acceptance in the clinical contexts described above, presumably due to the high effort for establishing completely new oxygen supply arrangements and due to the necessity of changing long-term habits of clinical personnel.

From the unpublished European Patent Application No. 14 180 666.1, a method of upgrading a clinical oxygen supply arrangement adapted to supply supplemental oxygen to a patient, comprising an oxygen wall outlet, a manually operable oxygen flow setting device coupled to the oxygen wall outlet, and an oxygen supply line coupled to the manually operable oxygen flow setting device and adapted to provide oxygen to a patient in need of oxygen supply is known. In this application, it is suggested to provide an oxygen saturation sensing device attachable to the patient and adapted to provide data indicative of an oxygen saturation of the blood of the patient on the basis of a non-invasive measurement thereof. Furthermore, it is suggested to substitute the manually operable oxygen flow setting device by an automatically operable oxygen flow setting device comprising control means adapted to control an oxygen flow through the automatically operable oxygen flow setting device on the basis of the data from the oxygen saturation setting device in an automatic operation mode of the automatically operable oxygen flow setting device.

While such an arrangement provides significant advantages over the prior art by allowing to simply substitute a manually operable oxygen flow setting device by an automatically operable oxygen flow setting device without further amending the oxygen supply infrastructure of a hospital or the like, there is still a need for further improvement, especially in view of an improved autarky of the oxygen flow setting device used in such an arrangement.

An object of the present invention is thus to provide an oxygen supply device that is simple, reliable, adapted for autarkic operation and suitable to be used as a substitute of classical oxygen supply arrangements or parts thereof.

### SUMMARY OF THE INVENTION

According to the invention, a clinical oxygen supply device, a clinical oxygen supply system, and a method of clinical oxygen supply according to the independent claims is provided. Preferred embodiments of the invention are subject of the dependent claims and of the description that follows.

As mentioned before, the present invention is used in oxygen supply arrangements wherein a patient is supplied with supplemental oxygen directly from an oxygen wall outlet via an oxygen flow setting device, e.g. by use of a laryngeal mask or a nasal cannula, but without the use of sophisticated ventilators that completely substitute autonomous breathing. The invention thus relates to cases wherein an oxygen flow to the patient is directly influencable via regulation of the oxygen flow setting device, while in ventilators the ventilator itself regulates the oxygen flow. The terms “oxygen flow setting device” and “oxygen supply device” are used interchangeably herein.

Conventionally, as mentioned, in such scenarios a mechanical oxygen flow setting device is used. Such a mechanical oxygen flow setting device, as mentioned, may be substituted by an automatically operable oxygen flow setting device. However, the automatic operation of such an automatically operable oxygen flow setting device is generally based on electric energy which, according to the prior art, needs to be provided via either a mains, i.e. via an electrical connection to a supply system of electric energy, or via suitable energy storing devices such as batteries. This is the case for all oxygen flow setting devices that are not entirely mechanical but e.g. regulate the oxygen flow via electric means or provide a digital indication of an oxygen flow, even if no feedback from an oxygen saturation measurement is used.

When using batteries in such devices, however, a disruption in oxygen supply may occur if the batteries are discharged. While this problem may be addressed by providing a warning system informing the patient or clinical personnel of an imminent power failure, a corresponding warning could be ignored, especially when issued during times of restricted attendance, e.g. during weekends. Furthermore, if rechargeable batteries are used, they may be suffer from a fatigue effect due to frequent and/or improper recharging cycles resulting from continuous use. In consequence, to provide sufficient security against power losses, battery packs used in an oxygen flow setting device as mentioned tend to be rather bulky or, if high-capacity batteries are used, expensive. Bulky batteries also may, due to their weight, also exert significant mechanical forces upon an oxygen wall outlet and, at least over time, damage the latter. A rather frequent exchange such battery packs, furthermore, which may be inevitable due to the continuous demand for the oxygen flow setting device, may generate significant waste and therefore environmental problems.

In case an oxygen flow setting device requiring electrical energy is coupled to the mains, it is necessary to use electrical sockets which may not be provided in proximity to the oxygen wall outlet used to supply oxygen to the oxygen flow setting device. Therefore, typically rather unhandy electrical connections have to be made, including cables which may pose tripping and/or disconnection hazards.

The present invention overcomes these problems and suggests a conversion of pressure energy latently present in oxygen supplied to the oxygen supply device into electric energy that may be used to operate the oxygen supply device or the means for controlling an oxygen flow contained in such a device.

The present invention is especially based on the previous finding that an exchange of only the existing mechanical oxygen flow setting devices as described above to oxygen flow setting devices that are specifically adapted to be exchangeable therewith, leaving the rest of the clinical oxygen supply arrangement untouched, will provide broader acceptance of clinical oxygen supply arrangements provided thereby, and, consequently, allow for a better oxygen supply to patients which is more reliable and especially less prone to human error.

Conventionally, in the entirely manual method as described above, an operator of a oxygen flow setting device may, e.g. due to stress during the treatment of emergency cases or surgical treatment, omit to upregulate the oxygen flow or, in some cases with even more severe consequences, forget to downregulate a previously upregulated oxygen flow, thus providing excessive oxygen amounts to the patient. This can be avoided according to an embodiment of the present invention wherein a feedback from an oxygen saturation measurement of the blood of the patient is used in regulating the oxygen flow. The advantages of such an embodiment are explained below. However, as mentioned, for patient groups less sensitive to excessive oxygen, it may also be sufficient to only provide a fixed oxygen flow without feedback from a measurement. In such cases, the oxygen flow setting device may also be adapted to set the oxygen flow based on a specific user request indicating a desired flow only. Such an oxygen flow setting device then closely resembles, as far as operation is concerned, a classical rotameter and can thus easily exchanged therewith. However, it has significant advantages over a classical rotameter in terms of precision and reliability.

Conventionally, during the treatment of emergency cases or surgical treatment, the paradigm “as much oxygen as possible” was followed. More modern methods of treatment, however, suggest supplying “titrated oxygen”, i.e. provide optimum oxygen saturation, especially in patient groups which are extremely sensitive to excessive oxygen supply, e.g. patients with chronical obstructive lung disease (COPD). In such patients, excessive oxygen supply might result in reduced breathing or the complete stop of breathing. In case of myocardial infarction, seizures or stroke, a high peripheral blood perfusion is needed. Excessive oxygen supply, however, results in the reduction of peripheral blood flow and is therefore contraindicated.

Especially in these modern methods of treatment with tight oxygen titration, a manual operation of entirely manually operable oxygen flow setting devices becomes difficult or even impossible in the hectic environment of a hospital, since it would take time. It is highly likely that an operator, in an entirely manually operation, leaves the optimum range of the oxygen supply for specific types of patients. Completely exchanging existing clinical supply arrangements for establishing improved and automatically controllable systems would however include, as mentioned, high invest costs and/or a change of long-established habits in clinical practice.

The present invention further improves the convenience of a shift between oxygen flow setting devices by increasing the autarky of the devices involved. An oxygen flow setting device provided according to the present invention is significantly less bulky and less prone to power losses (as compared to an oxygen flow setting device including batteries) and/or of significantly less hindrance to clinical personnel and/or patients (as compared to an oxygen flow setting device connected to the mains). It thus much closer resembles or even exceeds, in terms of ease of operation, the mechanical and manually operable oxygen flow setting devices including rotameters which are classically used. Therefore, much broader acceptance in clinical contexts is to be expected, leading to an increase in general patient safety.

### Advantages of the invention

In view of the above, the present invention provides a clinical oxygen supply device adapted to supply supplemental oxygen to a patient. In other words, the present invention does, as initially mentioned, not relate to scenarios wherein rather advanced oxygen supply units like ventilators are used, but rather to scenarios wherein patients are supplied with oxygen to support and/or improve autonomous breathing.

The clinical oxygen supply device according to the present invention comprises an oxygen inlet section with a first coupling unit adapted to be releasably coupled to a standardized oxygen wall outlet. Via such a first coupling unit, which may also be provided as an exchangeable unit and therefore be adaptable to different oxygen wall outlet standards, the oxygen supply device according to the present invention may be coupled easily to a number of different oxygen supply systems and/or their respective wall outlets. It is also possible to provide a clinical oxygen supply device with different coupling units that are supplied as exchange and/or spare parts. In a particularly preferred embodiment, the coupling unit is also adapted to be releasably coupled to an oxygen cylinder, e.g. via a suitable adapter. In this way, e.g. a relocation of a patient from one ward to another or from one hospital to another becomes possible without significant oxygen supply breaks. During transport, the oxygen coupling unit is then releasably coupled to the oxygen cylinder for an nearly uninterrupted oxygen supply.

According to the present invention, the clinical oxygen supply device further includes an oxygen outlet section with a second coupling unit which is adapted to be releasably coupled to an oxygen dispensing device which is attached or attachable to a patient. Oxygen dispensing devices useable in the context of the present invention include, for example, nasal cannulas, facial (laryngeal) masks and further units as mentioned. As also mentioned, the context of the present invention is rather the supplemental oxygen supply. Thus, the oxygen dispensing device which is attached or attachable to the patient is typically not embodied as an intubation unit. However, also intubation units are intended to be encompassed by the scope of the present invention.

The clinical oxygen supply device of the present invention further comprises an oxygen flow control section. This oxygen flow section is provided to restrict an oxygen flow between the oxygen inlet section and the oxygen outlet section in order to provide a controlled oxygen flow to the patient. As explained below, the oxygen flow control section may also be supplied with data regarding a status and/or an oxygen need of the specific patient, e.g. according to a treatment protocol provided from externally and/or stored in storage means of the oxygen flow control section. The oxygen flow control section of the clinical oxygen supply device according to the present invention includes a regulating unit adapted to restrict the oxygen flow between the oxygen inlet section and the oxygen outlet section in a regulated manner in order to provide the controlled oxygen flow to the patient. The regulating unit is also adapted, according to the present invention, to operate on the basis of a control signal in order to provide the controlled oxygen flow to the patient.

As mentioned, the clinical oxygen supply device of the present invention may be adapted to operate on the basis of a flow setting from a user, i.e. the user may enter a desired flow of oxygen. In this case, the control signal to the regulating unit may be based on a flow setting. The flow setting may be defined by a user, e.g. via a user interface unit, or via stored data. For example, for a specific user or user group or specific treatment targets, a desired oxygen flow may be defined, stored, and later be recalled from memory in order to provide the control signal to the regulating unit. A user interface unit may include appropriate setting means, e.g. buttons, keypads, touchscreens, control knobs, etc.

According to a further embodiment of the present invention, as mentioned, the clinical oxygen supply device may also operate on the basis of a measurement feedback. In this embodiment, the clinical oxygen supply device comprises a communication unit which is adapted to receive data from an oxygen saturation sensing device which is also attached to the patient. The oxygen saturation sensing device is adapted to measure, via a typically non-invasive measurement, an oxygen saturation of the blood of the patient, according to the principles as mentioned above. In this embodiment, the control signal to the regulating unit is provided on the basis of the data from the oxygen saturation sensing device received via the communication unit.

According to the present invention, to circumvent the problems as indicated above, the clinical oxygen supply device comprises an energy conversion section which is adapted to generate electrical energy from pressure energy of oxygen that is supplied to the oxygen inlet section. Furthermore, according to the present invention, an energy supply section is provided which is adapted to provide the electrical energy generated by the energy conversion section to the oxygen flow control section. As mentioned above, the present invention is essentially based on the finding that conversion of latent energy contained in the oxygen that is supplied to the clinical oxygen supply device may be used to generate electric energy and to provide a more reliable and/or convenient energy supply to the clinical oxygen supply device and/or its components. Regarding specific advantages of the solution according to the present invention, reference is made to the explanations above.

According to the present invention, display means are provided which are adapted to indicate at least one operating parameter of the clinical oxygen supply device. For example, such display means may indicate the controlled oxygen flow, e.g. in liters per minute, provided by the oxygen flow control section. Such display means may, however, also be adapted to display other parameters, e.g. oxygen pressure, patient data or an oxygen saturation in the blood of the patient, if measured. The display means are also adapted to be supplied with electrical energy by the energy supply section and vice versa.

Further according to the present invention, the clinical oxygen supply device is a battery-less device and is especially configured in a way that an amount of electrical energy provided by the energy conversion section corresponds, in all operation modes of the clinical oxygen supply device, to the amount of electrical energy supplied by the energy supply section. In other words, if the energy conversion section does not provide electrical energy, e.g. because the clinical oxygen supply device is not connected to a source of oxygen, also the energy supply system does not supply electrical energy. An “amount of electrical energy”, in this context, especially corresponds to an electrical current or power which is provided by the energy conversion section and which is directly used and supplied by the energy supply section, especially per unit time.

As a battery-less device, the clinical oxygen supply device can be configured without complicated means for charging and controlling batteries and can therefore be constructed much simpler and especially lighter as compared to devices according to the prior art which comprise batteries. The clinical oxygen supply device does, despite being a battery-less system, provide the same security of oxygen supply like conventional devices for oxygen supply because it is provided as a device for coupling to a wall outlet, as mentioned.

In hospitals where oxygen is typically provided from a central tank to numerous wall outlets, a constant oxygen supply must be safeguarded at any time by centrally controlling the oxygen supply to the wall outlets. In other words, it can be expected, from a consumer point of view, that there is always a sufficient oxygen pressure present at the wall outlet. In yet other words, the wall outlets can be defined as reliable and uninterrupted oxygen sources. Therefore, if oxygen from the wall outlet is used as the source of energy in a clinical oxygen supply device mounted to a wall outlet, no additional sources of electrical energy or storage means like batteries are needed.

For short times during which the patient is e.g. transferred between wards, the clinical oxygen supply device can be operated manually as in such times clinical personnel is present anyway. Instead of providing batteries only for such short time intervals, which are simply unnecessary when the clinical oxygen supply device is connected to a reliable wall outlet, according to the present invention batteries are totally dispensed with. This also eliminates a source of error or failure, e.g. of battery control systems.

While generating electrical energy from a stream of oxygen to charge a battery is known in the context of oxygen supply, e.g. from U.S. Pat. No. 5,370,112 A, WO 2011/003339 A1 or US 2012/1234323 A1, the skilled person would not have contemplated to dispense with a battery in such devices.

As the oxygen supply device according to the present invention becomes significantly lighter and less bulky, its weight can be carried by an oxygen wall outlet alone without the need of further support structures. Its handling is therefore significantly improved and no floor or wall space is lost for further fixture elements. This represents a particular advantage in clinical contexts where space is often limited due to the presence of a plurality of further treatment devices. If the weight of the oxygen supply device is carried by the oxygen wall outlet and if no further support structures are necessary, also the blocking of neighbouring oxygen wall outlets by such support structures is avoided. In its simplest embodiment, the oxygen supply device according to the present invention resembles an “automatic rotameter” in which, when the oxygen is turned on at the wall outlet the device is connected to, electrical energy is generated. After thereby activating the oxygen supply device, an oxygen flow can be set, e.g. via a user interface unit as indicated above, and on this basis a control signal to the regulation unit is provided. The oxygen supply device may, in a basic embodiment, indicate only the set oxygen flow via the display means and therefore provide essentially that information that also a classical rotameter provides. However, as mentioned, the oxygen supply device according to the present invention provides, even in this basic embodiment, precision advantages and extensibility options. For example, essentially by way of a “plug in” arrangement, sensor data can be utilized.

According to a particularly advantageous embodiment of the present invention, the energy conversion section comprises a first energy conversion unit which is adapted to convert the pressure energy of the oxygen supplied to the oxygen inlet section to mechanical energy, and a second energy conversion unit which is adapted to convert the mechanical energy into the electrical energy mentioned above. By providing these components, the present invention may make use of standardized products and/or components, as e.g. used in pneumatic technology. The clinical oxygen supply device according to the present invention, therefore, allows for a particularly reliable operation by using standardized and well-tested equipment.

It is particularly advantageous if the first energy conversion unit used in the energy conversion section of the clinical oxygen supply device according to the present invention is provided as a pneumatic motor and/or an expansion turbine which is adapted to be operated by expansion of the oxygen supply by the oxygen inlet section. Especially pneumatic motors are well-tested and reliable components used in pneumatic technology. A pneumatic motor is a type of motor which provides mechanical energy by expanding a compressed fluid. While pneumatic motors are, in classical pneumatic technology, typically operated by expanding air, they are likewise usable for expanding oxygen and/or oxygen-rich fluids.

It should be noted that, if the present application uses the term “oxygen”, this term also includes oxygen-rich fluids which do not entirely consist of oxygen. The term “oxygen” according to the present application, therefore, also includes fluids or breathing gases which are enriched in oxygen, “enriched” meaning an oxygen content which is significantly above the oxygen content of atmospheric air, i.e. above 25%, 50% and/or 75% by volume of oxygen.

Pneumatic motors are generally adapted to convert pressure energy to mechanical energy by generating either linear or rotary motion. Linear motion may be generated by using e.g. a diaphragm or piston actuator, while rotary motion may be generated by for example a vane type air motor or a piston air motor. To convert linear motion to rotational motion, if necessary, units well-known in the art may be used. Expansion turbines which may also be used as the first energy conversion unit according to the present invention are well-known to the skilled person.

It is further advantageous if the second energy conversion unit used in the energy conversion section of the clinical oxygen supply device according to the present invention is provided as an electric generator which is adapted to be rotated or otherwise actuated by the mechanical energy provided by the first energy conversion unit. The generator (also referred to as “alternator”) according to the present invention may, for example, be embodied as a linear or a rotational generator. If, for example, a pneumatic motor generating a linear motion as explained above is used as the first energy conversion unit, the second energy conversion unit may be embodied as a linear generator. Linear generators are commonly known to the skilled person and typically resemble linear motors which are operated to generate electric energy from mechanical energy instead of generating mechanical energy from electric energy. If, on the other hand, the first energy conversion unit is provided as a pneumatic motor and/or an expansion turbine generating rotational motion, the second energy conversion unit is preferably provided as a rotational electric generator. In both cases, a suitable mechanic coupling, i.e. suitable shafts and/or rods are provided between the first energy conversion unit and the second energy conversion unit of the energy conversion section of the clinical oxygen supply device according to the present invention.

It is particularly advantageous if the energy supply section comprises energy status information means which are adapted to inform a user, especially via the display means, regarding an amount of electrical energy generated by the energy conversion section and/or available to the energy supply section. For example, the energy states information means may also provide information to a user regarding the amount of electrical energy presently generated, this amount relating directly to pressure to the oxygen supply to the clinical oxygen supply device. If, for example, this electrical energy is below a certain threshold, it may be concluded that the oxygen pressure is too low, probably also for a sufficient supply to the patient. Therefore, the energy status information means may also serve as an indication of the oxygen pressure. They may thus function as a second (redundant) security instance, besides a pressure sensor which is separately provided. The energy status information means, due to the functions as explained above, may also serve as the only pressure sensing unit, allowing for a simpler construction of the clinical oxygen supply device according to the present invention.

According to a further advantageous embodiment of the present invention, the energy supply section may further comprise an energy supply port which is adapted to supply the electric energy generated by the energy conversion section, or a part thereof, also to the oxygen saturation sensing device, if present. Such sensing devices are typically, but not necessarily, connected to the clinical oxygen supply device according to the present invention via sensing cables. It is therefore possible without using further cables to supply electric energy to such oxygen saturation sensing devices. Also an oxygen saturation sensing device may therefore be constructed in a less bulky form as no batteries have to be provided.

The present invention also relates to clinical oxygen supply system which includes a standardized oxygen wall outlet adapted to be supplied with pressurized oxygen, an oxygen dispensing device and an optionally an oxygen saturation sensing device attachable to a patient. According to the present invention, such an oxygen supply system includes an clinical supply device explained above.

Furthermore, the present invention relates to a method of clinical oxygen supply which includes the use of a clinical oxygen supply device as explained above and/or a clinical oxygen supply system as mentioned. Both the clinical oxygen supply system and the method of clinical oxygen supply provided according to the present invention take profit of the advantages mentioned above, to which, therefore, specific reference is made.

Further features and advantages of the oxygen flow setting device, of the oxygen supply system, and of the oxygen supply method according to embodiments of the present invention are summarized and discussed below.

As mentioned, the oxygen flow setting device according to the specific embodiment of the present invention as described above, i.e. the oxygen flow setting device operating on the basis of a measurement feedback, comprises a communication unit adapted to communicate at least with the oxygen saturation sensing device. Under a “communication”, according to the language used herein, any means of data transfer between two units is to be understood. Communication especially includes mono- and bidirectional data exchange. This means that also simply sending of data indicative of an oxygen saturation of the blood of the patient from the oxygen saturation sensing device attached to the patient to the oxygen flow setting device is a form of communication. A communication, according to the present invention, may be realized via different communication protocols, media and/or layers, e.g. via Bluetooth, USB connections, Ethernet, conventional serial links and the like. A “communication” may also include the supply of electric energy.

Most preferably, the communication of the oxygen flow setting device according to the corresponding embodiment of the present invention comprises the use of at least one receiver which is adapted to wiredly and/or wirelessly receive at least the data from the oxygen saturation sensing device which are indicative of the oxygen saturation of the blood of the patient. A corresponding receiver can, according to another embodiment of the invention, also be adapted to additionally or alternatively receive other data. At least one receiver may also be adapted to receive data from other units provided in a clinical setup, e.g. handheld devices adapted to input data, especially patient data nor treatment instructions, other oxygen supply arrangements and/or monitoring devices, as also explained below.

Correspondingly, the communication may also include the use of at least one sender which is adapted to wiredly and/or wirelessly send data to at least a monitoring unit and/or at least one other unit provided in the clinical setup. A monitoring unit provided according to a preferred embodiment of the present invention, which may also be provided independently from the communication unit and/or from the oxygen saturation sensing device, may be adapted to monitor the status of a number of oxygen flow setting devices and/or their electrical status.

Such a monitoring unit may especially be used to detect any malfunction and/or unexpected behaviour of an oxygen flow setting device and/or unexpected events relating to a status of one or more patients. Correspondingly, a preferred embodiment of the present invention includes that the data the at least one sender is adapted to send is indicative of a status of the patient and/or of the oxygen flow setting device. By providing a correspondingly adapted sender, it is possible for the clinical personnel to immediately react, e.g. in unexpected and/or emergency situations.

In this context, it is preferred that the oxygen flow setting device is further adapted to also be operated manually in at least one manual operation mode. A manual operation mode may for example be chosen if an unexpected status of the patient and/or of the oxygen flow setting device is registered by the monitoring unit described above. The monitoring unit as described above may in this case also be adapted to monitor a signal which continuously sent by the oxygen flow setting device. If such a signal, corresponding to e.g. a known dead-man's signal, is absent, the monitoring device may deduce that the flow setting device is unresponding and/or malfunctioning. In such cases, the monitoring device may issue a signal indicative of a malfunction of the oxygen flow setting device and instruct the personnel to perform a manual operation of the oxygen flow setting device.

In other words, it is preferred that the at least one manual operation mode is initiated if a determination is made that the oxygen flow setting device is malfunctioning and/or that the status of the patient is an unexpected one. Consequently, a non responsiveness and/or malfunctioning of the oxygen flow setting device may be detected early and an immediate action may be taken, minimizing the risk of harm to the patient.

As explained above, the determination that the oxygen flow setting device is malfunctioning is preferentially made on the basis of data sent by the flow setting device. Especially, if such data are implausible and/or cease to be sent, a corresponding determination may be made.

According to a preferred embodiment of the present invention, the oxygen flow setting device comprises a memory unit adapted to store patient and/or treatment data relating to the patient and/or a history of data provided by the oxygen saturation setting device, if provided. A corresponding memory unit may especially comprise a fixed or detachable memory device, e.g. fixed flash memory and/or a removable flash memory card or an USB key which may be inserted in a corresponding memory unit. On a corresponding memory device of the memory unit, the mentioned data may be stored, especially in encrypted form to meet privacy protection prerequisites. In such a memory unit and/or a corresponding memory device, the whole treatment history of the treatment of one or more patients may be stored and provided for later documentation. Possible treatment errors or unexpected events in the treatment may in this way be identified and provided as a basis for further treatment. It is also possible to send corresponding data to an external device, especially via the previously mentioned communication unit, to store such data in the external device for documentation and/or any other further use.

The oxygen flow setting device provided according to the present invention may especially include a proportional-integral-derivative (PIO) controller, as generally known in the field of oxygen supply.

The oxygen flow setting device provided according to the present invention may especially be adapted to store treatment data relating to the patient, e.g. including a specific oxygen demand of the patient to avoid excessive oxygen supply, as mentioned above.

According to a further preferred embodiment of the invention, the oxygen flow setting device may be adapted to correlate the patient and/or treatment data mentioned above with the data provided by the oxygen saturation sensing device indicative of the oxygen saturation of the blood of the patient. If e.g. a discrepancy between the latter and the patient and/or treatment data is identified and the discrepancy exceeds a predefined threshold for more than a predetermined time, a signal, as explained above, to a monitoring unit may be issued. In this case, the monitoring unit may initiate a manual operation of the oxygen flow setting device, leaving the ultimate decision on how to proceed to human skill.

Further advantages of the present invention are explained with reference to the appended drawings which illustrate an embodiment of the present invention.

### SHORT DESCRIPTION OF THE DRAWING

FIG. 1 shows an clinical oxygen supply device in a clinical oxygen supply system according to a preferred embodiment of the invention.

### EMBODIMENT OF THE INVENTION

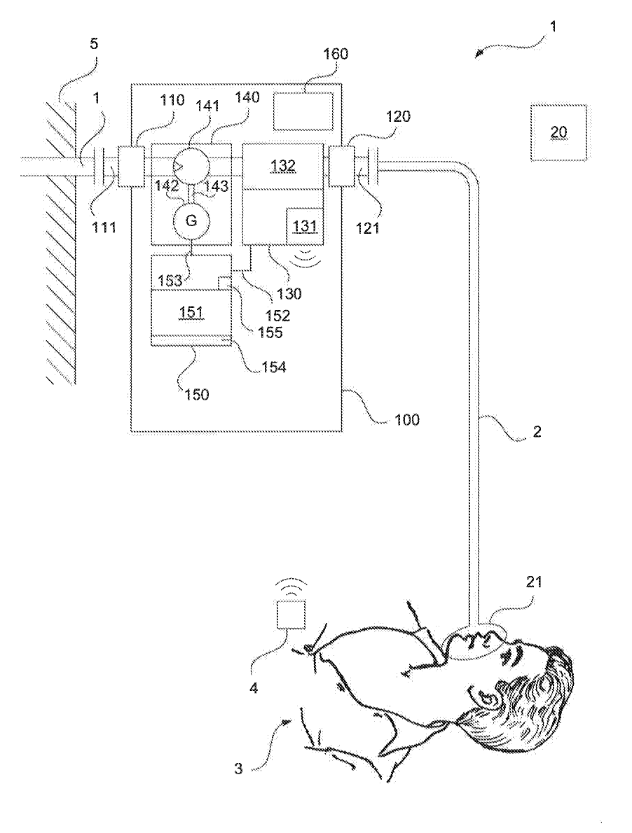

In FIG. 1, a clinical oxygen supply device according to an embodiment of the invention is indicated with 100. The clinical oxygen supply device 100 may form part of a clinical oxygen supply system 1, as explained above.

The clinical oxygen supply device 100 comprises an oxygen inlet section 110 adapted to receive oxygen and an oxygen outlet section 120 adapted to supply oxygen. The oxygen inlet section 110 comprises a first coupling unit 111 which is adapted to be releasably coupled to a standardized oxygen wall outlet 1, e.g. with suitable screw, push-fit, press-fit and/or bayonet coupling means. The oxygen wall outlet 1 is provided in a wall 5, e.g. of clinical ward.

The oxygen outlet section 120, on the other hand, comprises a second coupling unit 121 which is adapted to be releasably coupled to an oxygen dispensing device 2 which can, as shown in FIG. 1, be coupled to a patient 3. In the example shown, the oxygen dispensing device 2 comprises a facial mask 21 but other suitable means of oxygen supply to a patient 3 may be provided, e.g. a nasal cannula.

The patient 3 is further equipped with an oxygen saturation sensing device 4 which may operate according to the principles as explained above. In the example shown in FIG. 1, the oxygen saturation sensing device is wirelessly coupled to a communication unit 131 of an oxygen flow control section 130 of the oxygen supply device 100 of the specific embodiment shown in FIG. 1. However, also a wired connection is possible, such a wired connection also allowing an oxygen saturation sensing device to be supplied with electric energy from the clinical oxygen supply device 100.

As mentioned, the present invention is equally useful in clinical oxygen supply devices which do not operate on the basis of sensor feedback, in which case the oxygen saturation sensing device 4 is either not present or does not communicate with the clinical oxygen supply device. In such a case, the clinical oxygen supply device does either not comprise a communication unit 131 or the communication unit 131 is present but not adapted to communicate with an oxygen saturation sensing device 4, if present. The oxygen flow control section, of which the communication unit 131 is part of, is adapted to regulate an oxygen flow between the oxygen inlet section 110 and the oxygen outlet section 120 via suitable means not shown in detail. The oxygen flow is regulated by the regulation unit 132 on the basis of a control signal. In the example shown in FIG. 1, the control signal to the regulating unit 132 is provided on the basis of the data from the oxygen saturation sensing device 4 received by the communication unit 131, i.e. on the basis of an oxygen saturation of the blood of the patient 3 as measured by the oxygen saturation sensing device 4. However, as repeatedly mentioned, the control signal to the regulating unit 132 may be also based on a flow setting by a user.

The present invention, in the embodiment shown in FIG. 1, includes providing an energy conversion section 140 which is adapted to generate electric energy from pressure energy of oxygen supplied to the oxygen inlet section 110. For this purpose, the energy conversion section in the example shown comprises a first energy conversion unit 141 which may be, as mentioned, be embodied as a pneumatic motor. The first energy conversion unit 141 may be coupled, via a shaft 143, to a second energy conversion unit 142, e.g. an electrical generator. The clinical oxygen supply device 100 further includes an energy supply section 150 which is, e.g. via a energy supply line 152, adapted to provide electrical energy generated by the energy conversion section 140 to the oxygen flow control section 130. The energy supply section 150 may, via a further line 153, be connected to the energy conversion section 140. The energy supply section 150 would have to include an energy buffering unit 151 which would have to be adapted to buffer the electric energy generated by the energy conversion section 140 if the clinical oxygen supply device 100 were not adapted to be coupled to the oxygen wall outlet or coupled thereto. As the clinical oxygen supply device 100 is, however, in the context of the present invention, connected or connectable to such a reliable source of oxygen, the energy buffering unit 151, especially a battery, can be dispensed with. The energy buffering unit 151 is therefore only shown for reference purposes and does not form part of the embodiment of the invention. The energy supply section 150 may also comprise energy status information means 154, the function of which was already explained in detail above. An energy supply port may also form part of the energy supply section, the energy supply port being adapted to supply the electric energy generated by the energy conversion section 140 also to the oxygen saturation sensing device 5, if the latter is wiredly connected to the clinical oxygen supply device according to the present invention.

Furthermore, the clinical oxygen supply device may also include a manual operation section 160 which may be adapted to manually operate the oxygen flow control section 130 at least in case of a malfunction of, and/or a lack of oxygen and therefore electrical energy available to, the energy supply section 150, e.g. in case a patient is relocated. The manual operation section 160 may also be adapted to be supplied with clinical data, e.g. patient data regarding the status of the patient 3 and/or a treatment protocol. The manual operation section 160 may also be connected to further devices, e.g. via a wired connection or wirelessly. The clinical oxygen supply device 100 according to the embodiment shown may therefore also be remotely operable, e.g. from a central terminal and/or a corresponding unit which is indicated with 20. As mentioned, display means are provided according to the present invention. They can e.g. be part of the manual operation section 160 and may be powered from electrical energy from the energy supply section 150.

1. A clinical oxygen supply device adapted to supply supplemental oxygen to a patient, comprising an oxygen inlet section with a first coupling unit adapted to be releasably coupled to a standardized oxygen wall outlet, an oxygen outlet section with a second coupling unit adapted to be releasably coupled to an oxygen dispensing device attached to a patient, and an oxygen flow control section with a regulation unit adapted to regulate an oxygen flow between the oxygen inlet section and the oxygen outlet section on the basis of a control signal, characterized by display means adapted to indicate at least one operating parameter of the clinical oxygen supply device, an energy conversion section adapted to generate electrical energy from pressure energy of oxygen supplied to the oxygen inlet section, and an energy supply section adapted to provide the electrical energy generated by the energy conversion section to the oxygen flow control section and the display means, and further characterized in that the clinical oxygen supply device configured as a battery-less device.

2. A device according to claim 1, configured in a way that an amount of electrical energy provided by the energy conversion section corresponds, in all operation modes of the device, to an amount of electrical energy supplied by the energy supply section.

3. A device according to claim 1, wherein the oxygen flow control section comprises a user interface unit adapted to receive data from a user input and to provide the control signal to the regulation unit on the basis of the user input received by the user interface unit.

4. A device according to claim 1, wherein the energy conversion section comprises a first energy conversion unit adapted to convert the pressure energy of the oxygen supplied to the oxygen inlet section to mechanical energy and a second energy conversion unit adapted to convert the mechanical energy into the electrical energy.

5. A device according to claim 4, wherein the first energy conversion unit is provided as a pneumatic motor and an expansion turbine adapted to be operated by expansion of the oxygen supplied to the oxygen inlet section.

6. A device according to claim 4, wherein the second energy conversion unit is provided as an electric generator adapted to be operated by the mechanical energy provided by the first energy conversion unit.

7. A device according to claim 1, wherein the energy supply section comprises energy status information means adapted to inform a user regarding an amount of electrical energy generated by the energy conversion section and available to the energy supply section.

8. A device according to claim 1, further comprising a manual 15 operation section adapted to manually operate the oxygen flow control section at least in case of a malfunction of and a lack of electrical energy available to, the energy supply section.

9. A clinical oxygen supply system, comprising
a clinical oxygen supply device adapted to supply supplemental oxygen to a patient,
an oxygen inlet section with a first coupling unit adapted to be releasably coupled to a standardized oxygen wall outlet,
an oxygen outlet section with a second coupling unit adapted to be releasably coupled to an oxygen dispensing device attached to a patient,
an oxygen flow control section with a regulation unit adapted to regulate an oxygen flow between the oxygen inlet section and the oxygen outlet section on the basis of a control signal,
a display adapted to indicate at least one operating parameter of the clinical oxygen supply device, an energy conversion section adapted to generate electrical energy from pressure energy of oxygen supplied to the oxygen inlet section,
an energy supply section adapted to provide the electrical energy generated by the energy conversion section to the oxygen flow control and the display,
a standardized oxygen wall outlet adapted to be supplied with pressurized oxygen and an oxygen dispensing device, and
wherein the clinical oxygen supply device is configured as a battery-less device.

10. The system according to claim 9, further comprising an oxygen saturation sensing device attachable to a patient.

11. The system according to claim 10, wherein the oxygen flow control section further comprises a communication unit adapted to receive data from the oxygen saturation sensing device attached to the patient and to provide the control signal to the regulation unit on the basis of the data received from the oxygen saturation sensing device.

12. The system according to claim 11, wherein the energy supply section further comprises an energy supply port adapted to supply the electric energy generated by the energy conversion section also to the oxygen saturation sensing device.

13. A method of supplying clinical oxygen, comprising the steps of:
adapting a clinical oxygen supply device to supply supplemental oxygen to a patient,
adapting an oxygen inlet section with a first coupling to be releasably coupled to a standardized oxygen wall outlet,
adapting oxygen outlet section with a second coupling unit to be releasably coupled to an oxygen dispensing device attached to a patient,
adapting an oxygen flow control section with a regulation unit to regulate an oxygen flow between the oxygen inlet section and the oxygen outlet section on the basis of a control signal,
adapting a display to indicate at least one operating parameter of the clinical oxygen supply device, an energy conversion section adapted to generate electrical energy from pressure energy of oxygen supplied to the oxygen inlet section,
adapting an energy supply section adapted to provide the electrical energy generated by the energy conversion section to the oxygen flow control and the display,
adapting a standardized oxygen wall outlet adapted to be supplied with pressurized oxygen and an oxygen dispensing device, and
configuring the clinical oxygen supply device as a battery-less device.

14. The method according to claim 13, further comprising the steps of configuring the clinical oxygen supply device such that an amount of electrical energy provided by the energy conversion section corresponds, in all operation modes of the clinical oxygen supply device to an amount of electrical energy supplied by the energy supply section.

15. The method according to claim 14, further comprising the steps of adapting a user interface unit of the oxygen flow control section to receive data from a user input and to provide the control signal to the regulation unit on the basis of the user input received by the user interface unit.

16. The method according to claim 13, further comprising the steps of adapting a first energy conversion unit of the energy conversion section to convert the pressure energy of the oxygen supplied to the oxygen inlet section to mechanical energy and adapting a second energy conversion unit to convert the mechanical energy into the electrical energy.

17. A device according to claim 4, wherein the first energy conversion unit is provided as a pneumatic motor or an expansion turbine adapted to be operated by expansion of the oxygen supplied to the oxygen inlet section.

18. A device according to claim 5, wherein the second energy conversion unit is provided as an electric generator adapted to be operated by the mechanical energy provided by the first energy conversion unit.

19. A system according to claim 12, wherein the energy supply section comprises energy status information means adapted to inform a user regarding an amount of electrical energy generated by the energy conversion section or available to the energy supply section.

20. A system according to claim 12, further comprising a manual operation section adapted to manually operate the oxygen flow control section at least in case of a malfunction of or a lack of electrical energy available to the energy supply section.
